(19) United States
(12) Patent Application Publication
(10) Pub. No.: US 2026/0114819 A1
(43) Pub. Date: Apr. 30, 2026

(54) BIO-SIGNAL ESTIMATOR USING GENERATIVE ARTIFICIAL INTELLIGENCE MODEL

(71) Applicant: MEDIATEK INC., Hsinchu City (TW)

(72) Inventors: Shu-Yu Hsu, Hsinchu City (TW); Shih-Gu Huang, Hsinchu City (TW); Tzu-Yin Wei, Hsinchu City (TW)

(73) Assignee: MEDIATEK INC., Hsinchu City (TW)

(21) Appl. No.: 18/931,130

(22) Filed: Oct. 30, 2024

(51) Int. Cl. A61B 5/00; A61B 5/024; A61B 5/0245

(52) U.S. Cl. CPC A61B 5/7278; A61B 5/02416; A61B 5/0245; A61B 5/7221; A61B 5/7264; A61B 2560/0209

(57) ABSTRACT

A bio-signal estimator includes a bio-signal reader, a bio-signal monitor, a generative artificial intelligence (GAI) model, an integrated gate, and a power control. The bio-signal reader is used to read a plurality of input signals. The bio-signal monitor is linked to the bio-signal reader for generating a measured bio-signal according to the plurality of input signals. The GAI model is linked to the bio-signal reader and the bio-signal monitor for generating generated input signals and a generated bio-signal according to past input signals and past measured bio-signals generated by the bio-signal monitor. The integrated gate is used to generate a final bio-signal according to the generated input signals, the generated bio-signal and/or the measured bio-signal. The power control is used to turn on or turn off power of the bio-signal reader according to a first correctness probability and a second correctness probability of the GAI model.

### BACKGROUND

Wearable devices are designed to be used while worn. Common types of wearable devices include smartwatches and smartglasses. Wearable devices are often positioned close to or on the surface of the skin, where the wearable devices can detect, analyze, and transmit information such as bio-signals which allows immediate biofeedback to the wearer.

When using wearable devices, poor input signals are often caused by reasons such as wearing status and action status. Traditional algorithms can only handle short periods of input signal failure, and can easily introduce large errors over time. Therefore, a bio-signal estimator using generative artificial intelligence model is desired.

### SUMMARY

An embodiment provides a bio-signal estimator including a bio-signal reader, a bio-signal monitor, a generative artificial intelligence (GAI) model, an integrated gate, and a power control. The bio-signal reader is configured to read a plurality of input signals. The bio-signal monitor is linked to the bio-signal reader, and configured to generate a measured bio-signal according to the plurality of input signals. The generative artificial intelligence (GAI) model is linked to the bio-signal reader and the bio-signal monitor, and configured to generate generated input signals and a generated bio-signal according to past input signals and past measured bio-signals generated by the bio-signal monitor. The integrated gate is linked to the generative artificial intelligence (GAI) model and the bio-signal monitor, and configured to generate a final bio-signal according to the generated input signals, the generated bio-signal and/or the measured bio-signal. The power control is configured to turn on or turn off power of the bio-signal reader according to a first correctness probability of the GAI model and a second correctness probability of the GAI model.

Another embodiment provides a heart rate (HR) estimator including a heart rate reader, a generative artificial intelligence (GAI) model, an integrated gate, and a power control. The heart rate reader is configured to read a plurality of input signals. The heart rate monitor is linked to the heart rate reader, and configured to generate a measured heart rate according to the plurality of input signals by transforming the plurality of input signals to frequency domain. The generative artificial intelligence (GAI) model is linked to the bio-signal reader and the heart rate monitor, and configured to generate generated input signals and a generated heart rate according to past input signals and past measured heart rates generated by the heart rate monitor. The integrated gate is linked to the generative artificial intelligence (GAI) model and the heart rate (HR) monitor, and configured to generate a final heart rate (HR) according to the generated input signals, the generated heart rate and/or the measured heart rate. The power control is configured to turn on or turn off power of the heart rate reader according to a first correctness probability of the GAI model and a second correctness probability of the GAI model.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram of a heart rate (HR) estimator according to an embodiment of the present invention.

FIG. 2 is a block diagram of the GAI model according to an embodiment of the present invention.

FIG. 3 is a block diagram of a heart rate (HR) estimator according to an embodiment of the present invention.

FIG. 4 is the schematic diagram of the prediction of the GAI model in FIG. 2 according to an embodiment of the present invention.

### DETAILED DESCRIPTION

The terms using “heart rate” are not limited to heart rate. The heart rate estimator in the embodiment is an example of bio-signal estimator. All terms using “heart rate” can be replaced by “bio-signal”, and the apparatus and method in this invention are not limited to being used for heart rate but can be used for other bio-signals.

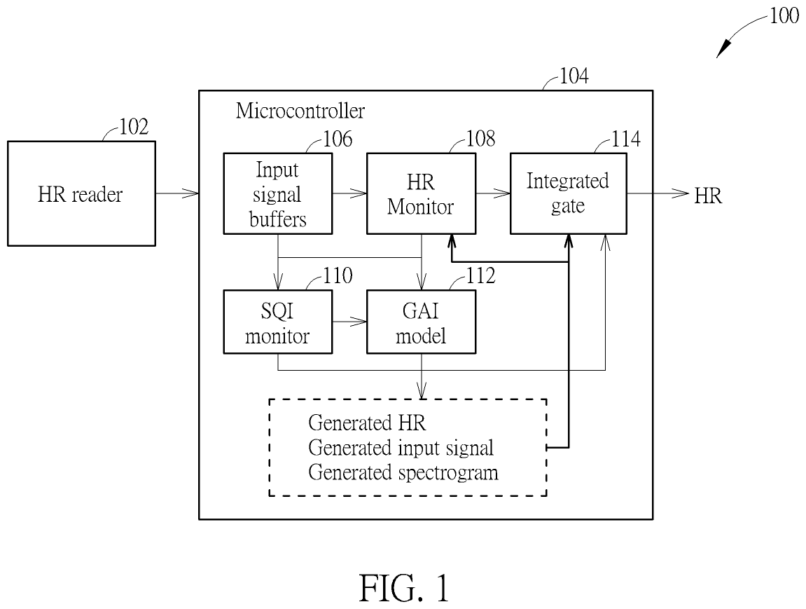

FIG. 1 is a block diagram of a heart rate (HR) estimator 100 according to an embodiment of the present invention. The heart rate estimator 100 includes a heart rate reader 102 and a microcontroller 104. The heart rate reader 102 is configured to measure input signals such as photoplethysmography (PPG), electrocardiogram (ECG) and acceleration and send them to the microcontroller 104. The microcontroller 104 includes a plurality of input signal buffers 106, a heart rate (HR) monitor 108, a signal quality indicator (SQI) monitor 110, a generative artificial intelligence (GAI) model 112 and an integrated gate 114.

The plurality of input signal buffers 106 are configured to store a plurality of input signals. The heart rate monitor 108 is linked to the plurality of input signal buffers 106, and configured to generate a measured heart rate according to the plurality of input signals. The heart rate monitor 108 transforms the input signals to a frequency domain and generates the measured heart rate from the frequency domain. In some other embodiments, the heart rate monitor may generate the measured heart rate based on the input signal, information related to the frequency domain, or any combination thereof. The SQI monitor 110 is linked to the plurality of input signal buffers 106 and the heart rate monitor 108, and configured to generate a signal quality according to the plurality of input signals, the measured heart rate or any combination thereof. The GAI model 112 is linked to the plurality of input signal buffers 106 and the heart rate monitor 108, and configured to generate generated input signals and a generated heart rate according to past input signals in the plurality of input signal buffers 106 and past measured heart rates generated by the heart rate monitor 108. For example, the past input signals are last five or other number of samples of input signals generated from the heart rate reader 102. The past measured heart rates are last five or other number of samples of measured heart rate generated by the heart rate monitor 108. The integrated gate 114 is linked to the GAI model 112, the heart rate monitor 108 and the SQI monitor 110, and configured to generate a final heart rate according to the generated input signals, the generated heart rate, the measured heart rate and/or the signal quality.

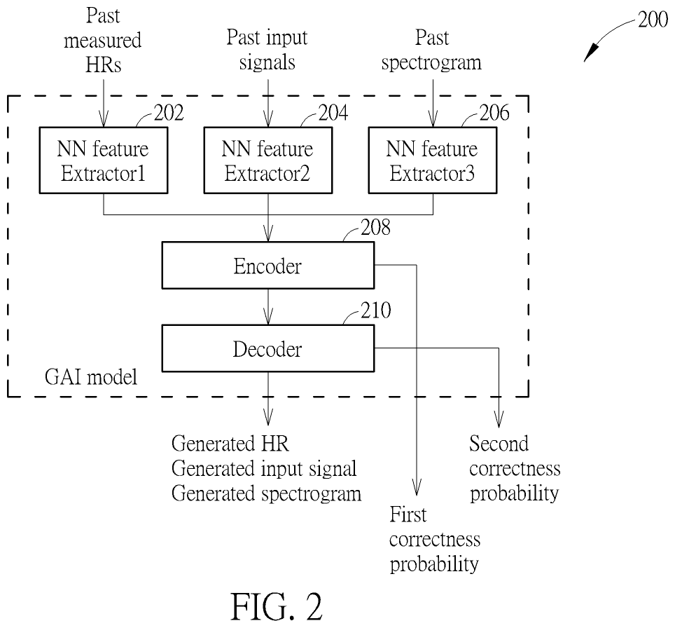

FIG. 2 is a block diagram of the GAI model 200 according to an embodiment of the present invention. The GAI model 200 includes a plurality of neural network (NN) feature extractors 202, 204, 206, an encoder 208 and a decoder 210. The plurality of neural network feature extractors 202, 204, 206 generate a plurality of extracted signals according to at least one of the past input signals, the past measured heart rates, a past spectrogram or combination thereof. In FIG. 2, the past measured heart rates are fed into a first NN feature extractor 202, the past input signals are fed into a second NN feature extractor 204, and the past spectrogram is fed into a third NN feature extractor 206. The three NN feature extractors output extracted signals to the encoder 208, and the encoder 208 generates a plurality of low level signals and a first correctness probability of the generative artificial intelligence (GAI) model 200 according to the plurality of extracted signals. The decoder 210 generates a plurality of high level signals and a second correctness probability of the generative artificial intelligence (GAI) model 200 according to the plurality of low level signals. The first correctness probability and the second correctness probability represent the probability of correctness of the GAI model 200 in this prediction. The high level signals include generated heart rates, generated input signals, and a generated spectrogram, thus predicting the future status of the heart rate (HR) estimator 100.

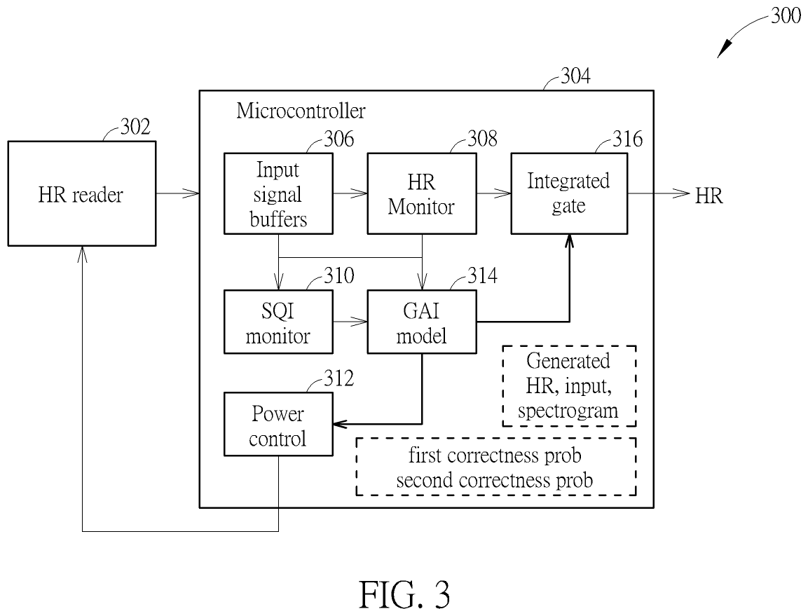

FIG. 3 is a block diagram of a heart rate (HR) estimator 300 according to an embodiment of the present invention. The heart rate estimator 300 includes a heart rate reader 302 and a microcontroller 304. The heart rate reader 302 is configured to measure input signals such as photoplethysmography (PPG), electrocardiogram (ECG) and acceleration them to the microcontroller 304. The microcontroller 304 includes a plurality of input signal buffers 306, a heart rate (HR) monitor 308, a signal quality indicator (SQI) monitor 310, a power control 312, 10 generative artificial intelligence (GAI) model 314 and an integrated gate 316.

The plurality of input signal buffers 306 are configured to store a plurality of input signals. The heart rate monitor 308 is linked to the plurality of input signal buffers 306, and configured to generate a measured heart rate according to the plurality of input signals. The SQI monitor 310 is linked to the plurality of input signal buffers 306 and the heart rate monitor 308, and configured to generate a signal quality according to the plurality of input signals and the measured heart rate. The GAI model 314 is linked to the plurality of input signal buffers 306 and the heart rate monitor 308, and configured to generate generated input signals, a generated heart rate, and a generated spectrogram according to past input signals in the plurality of input signal buffers 306 and past measured heart rates generated by the heart rate monitor 308. The power control 312 is linked to the GAI model 314, and configured to turn on or turn off the heart rate reader 302 according to the first correctness probability and the second correctness probability generated from the GAI model. The heart rate reader 302 can be turned off to save power or turn on to receive new input signals according to the correctness of GAI model prediction. If the first correctness probability is higher than a first threshold and/or the second correctness probability is higher than a second threshold, then the heart rate reader 302 can be turned off. If the first correctness probability is lower than a third threshold and/or the second correctness probability is lower than a fourth threshold, then the heart rate reader 302 should be turned on. The first threshold is larger than the third threshold. The second threshold is larger than the fourth threshold. The integrated gate 316 is linked to the GAI model 314, the heart rate monitor 308 and the SQI monitor 310, and configured to generate a final heart rate according to the generated input signals, the generated heart rate, the measured heart rate and/or the signal quality.

The heart rate monitor 308 generates the measured heart rate by transforming the input signals to frequency domain. The integrated gate 316 generates the final heart rate based on the plurality of high level signals generated by the decoder 210 and the measured heart rate generated by the heart rate monitor 308 using linear or nonlinear combination.

For linear combination, a weight is generated according to the signal quality, the first correctness probability and the second correctness probability, and the final heart rate can be generated by summing a product of the measured heart rate and the weight, and a product of the generated heart rate and a difference of 1 and the weight as follows:

$$final HR = weight \times ( measured HR ) + ( 1 - weight ) \times ( generated HR )$$

For nonlinear combination, a weight is generated according to the signal quality, the first correctness probability and the second correctness probability, and the final heart rate is the measured heart rate if the weight is larger than a predetermined threshold. The final heart rate is the generated heart rate if the weight is smaller than the predetermined threshold.

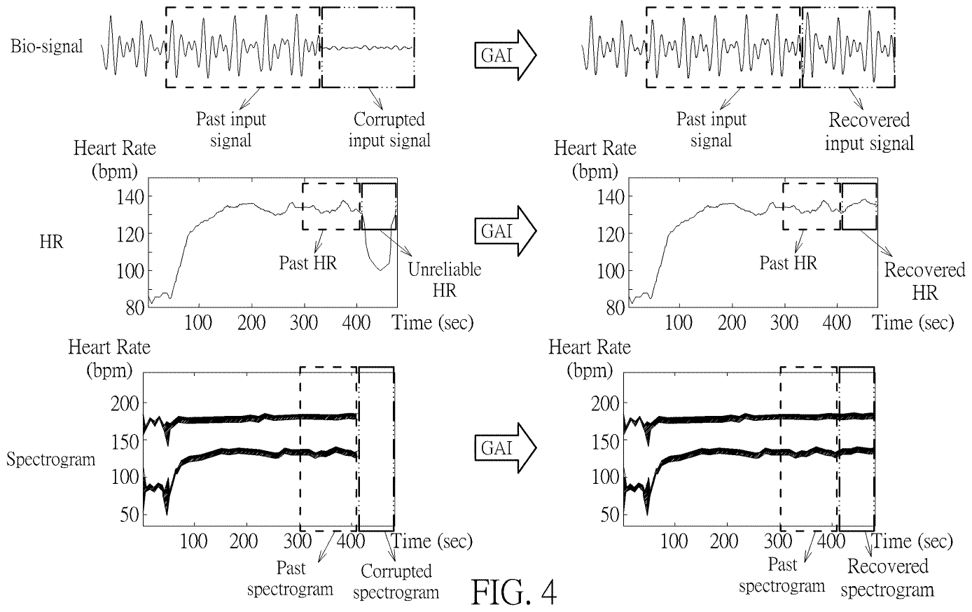

FIG. 4 is the schematic diagram 400 of the prediction of the GAI model 200 according to an embodiment of the present invention. In FIG. 4, the GAI model 200 recovers the input signal, the heart rate, and the spectrogram according to the past input signals, past heart rates, and the past spectrogram. The recovered input signal, the recovered heart rate, and the recovered spectrogram are more reliable than the corrupted input signals, the unreliable heart rates, and the corrupted spectrogram respectively. The heart rate estimator 100, 300 can handle large periods of input signal failure, and introduce small errors over time, thus can significantly improve the reliability of wearable devices.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A bio-signal estimator, comprising:
a bio-signal reader configured to read a plurality of input signals;
a bio-signal monitor configured to generate a measured bio-signal according to the plurality of input signals;
a generative artificial intelligence (GAI) model configured to generate generated input signals and a generated bio-signal according to at least one of past input signals and past measured bio-signals generated by the bio-signal monitor;
an integrated gate configured to generate a final bio-signal according to the generated input signals, the generated bio-signal and/or the measured bio-signal; and
a power control configured to turn on or turn off power of the bio-signal reader according to a first correctness probability of the GAI model and a second correctness probability of the GAI model.

2. The bio-signal estimator of claim 1, wherein the generative artificial intelligence (GAI) model comprises:
a plurality of neural network feature extractors configured to generate a plurality of extracted signals according to the past input signals, the past measured bio-signals and a past spectrogram;
an encoder configured to generate a plurality of low level signals and the first correctness probability of the GAI model according to the plurality of extracted signals; and
a decoder configured to generate a plurality of high level signals and the second correctness probability of the GAI model according to the plurality of low level signals.

3. The bio-signal estimator of claim 2, wherein the plurality of high level signals include generated bio-signals, generated input signals, and a generated spectrogram.

4. The bio-signal estimator of claim 2, wherein the integrated gate generates the final bio-signal based on the plurality of high level signals generated by the decoder and the measured bio-signal using linear or nonlinear combination.

5. The bio-signal estimator of claim 2, further comprising:
a plurality of input signal buffers configured to store the plurality of input signals and the past input signals.

6. A heart rate (HR) estimator, comprising:
a heart rate reader configured to read a plurality of input signals;
a heart rate monitor configured to generate a measured heart rate according to the plurality of input signals by transforming the plurality of input signals to frequency domain;
a generative artificial intelligence (GAI) model configured to generate generated input signals and a generated heart rate according to past input signals and past measured heart rates generated by the heart rate monitor;
an integrated gate configured to generate a final heart rate (HR) according to the generated input signals, the generated heart rate and/or the measured heart rate; and
a power control configured to turn on or turn off power of the heart rate reader according to a first correctness probability of the GAI model and a second correctness probability of the GAI model.

7. The heart rate (HR) estimator of claim 6, further comprising:
a plurality of input signal buffers configured to store the plurality of input signals and the past input signals.

8. The heart rate (HR) estimator of claim 7, further comprising:
a signal quality indicator monitor configured to provide a weighting function to the integrated gate for linearly or nonlinearly combining the generated heart rate and the measured heart rate.

9. The heart rate (HR) estimator of claim 8, wherein linearly combining the generated heart rate and the measured heart rate is implemented by summing a product of the measured heart rate and a weight, and a product of the generated heart rate and a difference of 1 and the weight.

10. The heart rate (HR) estimator of claim 8, wherein nonlinearly combining the generated heart rate and the measured heart rate is implemented according to a weight and a predetermined threshold.

11. The heart rate (HR) estimator of claim 10, wherein if the weight is larger than the predetermined threshold, then the final heart rate is the measured heart rate.

12. The heart rate (HR) estimator of claim 10, wherein if the weight is smaller than the predetermined threshold, then the final heart rate is the generated heart rate.

13. The heart rate (HR) estimator of claim 6, wherein the generative artificial intelligence (GAI) model comprises:
a plurality of neural network feature extractors configured to generate a plurality of extracted signals according to the past input signals, the past measured heart rates and a past spectrogram;
an encoder configured to generate a plurality of low level signals and the first correctness probability of the generative artificial intelligence (GAI) model according to the plurality of extracted signals; and
a decoder configured to generate a plurality of high level signals and the second correctness probability of the generative artificial intelligence model according to the plurality of low level signals.

14. The heart rate (HR) estimator of claim 13, wherein the past input signals include past photoplethysmography (PPG), past electrocardiogram (ECG) and/or past acceleration.

15. The heart rate (HR) estimator of claim 13, wherein the plurality of high level signals include generated heart rates, generated input signals, and generated spectrogram.

16. The heart rate (HR) estimator of claim 13, wherein the integrated gate generates the final heart rate based on the measured heart rate and the high level signals generated by the decoder using linear or nonlinear combination.

17. The heart rate (HR) estimator of claim 6, wherein the power control turns off the power of the heart rate reader when the first correctness probability is larger than a first threshold and/or the second correctness probability is larger than a second threshold.

18. The heart rate (HR) estimator of claim 6, wherein the power control turns on the power of the heart rate reader when the first correctness probability is smaller than a third threshold and/or the second correctness probability is smaller than a fourth threshold.
